(19) United States
(12) Patent Application Publication
(10) Pub. No.: US 2026/0001046 A1
(43) Pub. Date: Jan. 1, 2026

(54) DEVICE FOR PROVIDING A SYNTHETIC GAS MIXTURE OF AT LEAST CARBON DIOXIDE AND HYDROGEN

(71) Applicant: MAN Energy Solutions SE, Augsburg (DE)

(72) Inventors: Harald STRICKER, Berlin (DE); Klaus BEHNKE, Schriesheim (DE)

(21) Appl. No.: 19/245,623

(22) Filed: Jun. 23, 2025

(30) Foreign Application Priority Data
Jun. 26, 2024 (DE) 10 2024 117 973.2

(51) Int. Cl. B01F 23/10; B01F 23/70; B01F 35/83

(52) U.S. Cl. CPC B01F 23/19; B01F 23/711; B01F 35/833

(57) ABSTRACT

A device providing a synthetic gas mixture, providing a mass flow of carbon dioxide at a first pressure level and a mass flow of hydrogen at a second pressure level, having a splitting device splitting the mass flow of carbon dioxide into a first and second part mass flow, a part mass flow turbine expanding the first part mass flow, a first mixing device mixing the expanded first part mass flow with the mass flow of hydrogen, a compression device compresses the carbon dioxide and hydrogen to a third pressure level, a bypass line that conducts the second part mass flow past the part mass flow turbine, the first mixing device and the compression device in the direction towards a second mixing device, to mix at the second mixing device the second part mass flow conducted via the bypass line with the mixture compressed by the compression device.

### BACKGROUND OF THE INVENTION

### 1. Field of the Invention

The disclosure relates to a device for providing a synthetic gas mixture of at least carbon dioxide and hydrogen in particular for producing synthetic fuels.

### 2. Description of Related Art

For the production of synthetic fuels, such as, for example, for producing methane or methanol, hydrogen on the one hand and carbon dioxide on the other hand are required. The mixture of at least hydrogen and carbon dioxide is also referred to as synthetic gas mixture, which has to be provided at a defined pressure level to a device in which ultimately synthetic fuel is produced.

Accordingly, devices for producing synthetic fuels, such as for example, methane or methanol, operate at a pressure level between 30 bar and 70 bar. A synthetic gas mixture, which comprises at least carbon dioxide and hydrogen, has to be provided at this pressure level.

The carbon dioxide is preferentially provided by a carbon dioxide provision device, at a first pressure level and the hydrogen is preferentially provided by a hydrogen provision device at a second pressure level.

The hydrogen provision device can be, for example, an electrolyser, which, using electric power generated from regenerative energy sources, provides hydrogen. This hydrogen provided by the hydrogen provision device is provided at a second pressure level.

The carbon dioxide is provided at the first pressure level, wherein the carbon dioxide provision device can be, for example, a CCS (carbon capture and storage) plant or a DAC (direct air capture) plant.

The first pressure level, at which the gaseous carbon dioxide is provided, is typically higher than the second pressure level at which the hydrogen is provided. Thus, it is possible that the second pressure level, at which the gaseous hydrogen is provided, corresponds to atmospheric pressure (1 bar), and that the first pressure level, at which the gaseous carbon dioxide is provided, amounts to 30 bar or more.

In particular, when hydrogen gas, starting at the second pressure level is to be compressed to a pressure level at which a device for producing synthetic fuels operates, the technical device expenditure of the device increases because of the low molecular mass of the hydrogen gas. In particular, a large number of compressor stages is required. Relatively much energy for compressing the hydrogen gas is also necessary.

### SUMMARY OF THE INVENTION

There is a need for providing the synthetic gas mixture of at least carbon dioxide and hydrogen with lower technical device expenditure and lower energy requirement at that pressure level, which is required in particular by a device for producing synthetic fuels.

Starting out from this, one aspect of the present invention is based on creating a new type of device for providing a synthetic gas mixture of at least carbon dioxide and hydrogen.

The device according to one aspect of the invention comprises a splitting device for splitting the mass flow provided to the device from at least carbon dioxide in a first part mass flow from at least carbon dioxide, a second part mass flow from at least carbon dioxide. The device according to one aspect of the invention, further, comprises a part mass flow turbine for expanding the first part mass flow of at least carbon dioxide. The device further comprises a first mixing device for mixing the first part mass flow expanded by the part mass flow turbine of at least carbon dioxide with mass flow of at least hydrogen provided by the device. The device further comprises a compression device for compressing the mixture provided by the first mixing device of at least carbon dioxide and hydrogen to a third pressure level. The device further comprises a bypass line, via which the second part mass flow of at least carbon dioxide can be directed past the part mass flow turbine, past the first mixing device and past the compression device in the direction of a second mixing device, in order to mix in the region of the second mixing device the second part mass flow conducted via the bypass line of at least carbon dioxide with the mixture of at least carbon dioxide and hydrogen compressed by the compression device.

The device according to one aspect of the invention for providing the synthetic gas mixture of at least carbon dioxide and hydrogen the mass flow of at least carbon dioxide provided by a carbon dioxide provision device is split into part mass flows. A first part mass flow is mixed for the expansion via the part mass flow turbine and subsequently in the region of the first mixing device with the gaseous hydrogen, provided in particular by a hydrogen provision device, in order to subsequently compress this mixture in the compression device.

By mixing the hydrogen gas provided to the device with the first part mass flow of the carbon dioxide provided to the device, a gas mixture is provided, which has a significantly higher molecular mass than hydrogen gas as such, so that this mixture of carbon dioxide and hydrogen can be compressed with lower technical device expenditure and lower costs by the compression device. For compressing the mixture of the hydrogen gas and the first part mass flow of the carbon dioxide, fewer compressor stages are needed than for compressing the hydrogen gas as such. Further, through the polytropic expansion of the first part mass flow upstream of the compression device a temperature level for the mixture of hydrogen and carbon dioxide to be compressed is provided, which makes possible compressing the mixture with low power requirement.

The second part mass flow of the carbon dioxide provided to the device in particular by the carbon dioxide provision device is directed via the bypass line, past the part mass flow turbine, past the first mixing device and past the compression device in the direction of the second mixing device in order to mix the second part mass flow downstream of the compression device with the mixture compressed in the compression device. This is also advantageous in order to keep the technical device expenditure and energy requirement in the region of the compression device as low as possible.

The device according to one aspect of the invention for providing a synthetic gas mixture of at least carbon dioxide and hydrogen allows an efficient provision of a synthetic gas mixture of at least carbon dioxide and hydrogen in particular for producing synthetic fuels with low energy costs and low technical device expenditure.

Preferentially, the device according to one aspect of the invention has a total mass flow turbine for expanding the entire mass flow of at least carbon dioxide provided to the device at a pressure level dependent on the pressure level in the bypass line. This also serves for providing a synthetic gas mixture at a defined pressure level with minimal technical device expenditure and low energy requirement.

Preferentially, the device according to one aspect of the invention comprises a heating device arranged upstream of the part mass flow turbine for heating the first part mass flow. This also serves for providing a synthetic gas mixture at a desired pressure level with preferably low technical device expenditure and preferably low energy costs.

### BRIEF DESCRIPTION OF THE DRAWINGS

Preferred further developments of the invention are obtained from the subclaims and the following description. Exemplary embodiments of the invention are explained in more detail by way of the drawing without being restricted to this.

There it shows:

FIG. 1: is a device for providing a synthetic gas mixture of at least carbon dioxide and hydrogen in particular for producing synthetic fuels; and

FIG. 2: is a further development of the device of FIG. 1.

### DETAILED DESCRIPTION OF THE PRESENTLY PREFERRED EMBODIMENTS

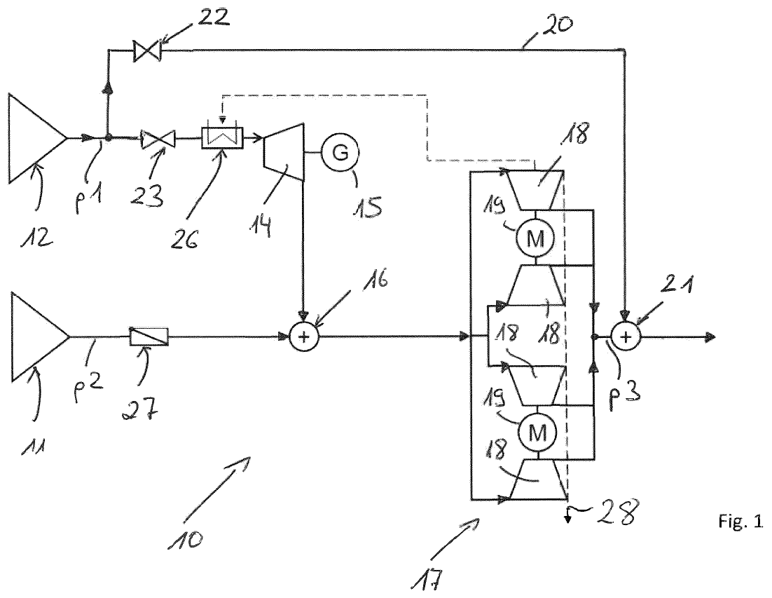

FIG. 1 shows in a highly schematised manner a block circuit diagram of a device 10 according to one aspect of the invention for providing a synthetic gas mixture of at least carbon dioxide and hydrogen in particular for producing synthetic fuels in a device for producing synthetic fuels which is not shown. In particular, a synthetic gas mixture is provided, which can be used for producing methane CH₄ or methanol CH₃OH.

The device 10 is provided a mass flow of at least carbon dioxide at a first pressure level p1 and a mass flow of at least hydrogen at a second pressure level p2.

In FIG. 1, a hydrogen provision device 11 provides at least gaseous hydrogen H₂ at the second pressure level p2. The hydrogen H₂ provided by the hydrogen provision device 11 can also contain water vapour H₂O and oxygen O₂. The hydrogen provision device 11 can be an electrolyser, which, using electric power generated from regenerative energy sources such as, for example, solar energy or wind energy, produces hydrogen.

In FIG. 1, a carbon dioxide provision device 12 provides gaseous carbon dioxide CO₂ at the first pressure level p1. The carbon dioxide provision device 12 can be a CCS plant or DAC plant.

The device 10 for providing a synthetic gas mixture of at least carbon dioxide and hydrogen according to the invention comprises a splitting device 13 in order to split, in the exemplary embodiment of FIG. 1, the mass flow provided to the device 10 at the first pressure level p1 of at least carbon dioxide into a first part mass flow and a second part mass flow.

A further constituent part of the device 10 for providing a synthetic gas mixture of at least carbon dioxide and hydrogen is a part mass flow turbine 14, which serves for the polytropic expansion of the first part mass flow of at least carbon dioxide. The first part mass flow is expanded in the turbine 14, wherein the turbine 14 drives a generator 15, which serves for generating electric energy.

The device 10 for providing a synthetic gas mixture of at least carbon dioxide and hydrogen, further, comprises a first mixing device 16, in which in the exemplary embodiment of FIG. 1, the first part mass flow expanded in the turbine 14 of at least carbon dioxide is mixed with the hydrogen provided to the device 10 at the second pressure level p2.

The device 10 for providing a synthetic gas mixture of at least carbon dioxide and hydrogen, furthermore, comprises a compression device 17, which serves for compressing the mixture provided by the mixing device 16 to a third pressure level p3.

FIG. 1 shows multiple parallel-connected compressors 18, wherein each two compressors 18 are driven by a common motor 19. The number of the compressors 18 in the compression device 17 is purely exemplary in nature. The interconnection of the compressors 18 in the compression device 17 is purely exemplary in nature as well. Accordingly, compressors 18 can also be interconnected in series.

The device according to one aspect of the invention 10 for providing a synthetic gas mixture of at least carbon dioxide and hydrogen furthermore comprises a bypass line 20, via which the second part mass flow of carbon dioxide split in the region of the splitting device 13 can be directed past the part mass flow turbine 14, past the first mixing device 16 and past the compression device 17 in the direction of a second mixing device 21, in order to mix the second part mass flow conducted in the region of the second mixing device 21 via the bypass line 20 with the mixture of the first part mass flow and the hydrogen compressed by the compression device 17. As already explained, the splitting device 13 serves for splitting the mass flow of at least gaseous carbon dioxide provided to the device 10 into the first part mass flow to be conducted via the part mass flow turbine 14 and the second part mass flow to be conducted via the bypass line 20.

The splitting device 13 comprises at least one valve 22, 23, wherein via the open position of the at least one valve 22, 23, the splitting of the mass flow of at least carbon dioxide provided to the device 10 into the two part mass flows can be adjusted. In FIG. 1, two valves 22, 23 of the splitting device 13 are shown. The splitting device can also comprise merely a single valve 22 preferentially in the region of the bypass line 20.

The first part mass flow can amount to between 25% and 75% and the second part mass flow to between 75% and 25% of the entire mass flow of at least carbon dioxide provided to the device 10, wherein the sum of both part mass flows corresponds to 100%.

It is also possible that the first part mass flow amounts to between 30% and 70% and the second part mass flow to between 70% and 30% or that the first part mass flow amounts to between 40% and 6% and the second part mass flow to between 60% and 40% of the entire mass flow of at least carbon dioxide provided to the device 10.

In particular, it is provided that a pressure level in the bypass line 20 is 0.1 bar to 1 bar higher than the third pressure level p3 of the mixture compressed in the compression device 17 directly downstream of the compression device 17 or in the region of the second mixing device 21.

In particular, the pressure in the bypass line 20 is between 0.1 bar and 0.6 bar or 0.1 bar and 0.5 bar or 0.2 bar and 0.6 bar or 0.2 bar and 0.5 bar higher than the third pressure level p3 of the mixture compressed in the compression device 17 directly downstream of the compression device 17 or in the region of the second mixing device 21.

In FIG. 1, merely pressure losses in the region of the respective valve arranged in the flow path and in the respective flow line occur between the splitting device 13 and the outlet point of the bypass line 20 into the second mixing device 21. From this it follows that the first pressure level p1, at which in FIG. 1 the carbon dioxide is provided, is slightly higher than the pressure level in the bypass line 20 directly downstream of the compression device 17.

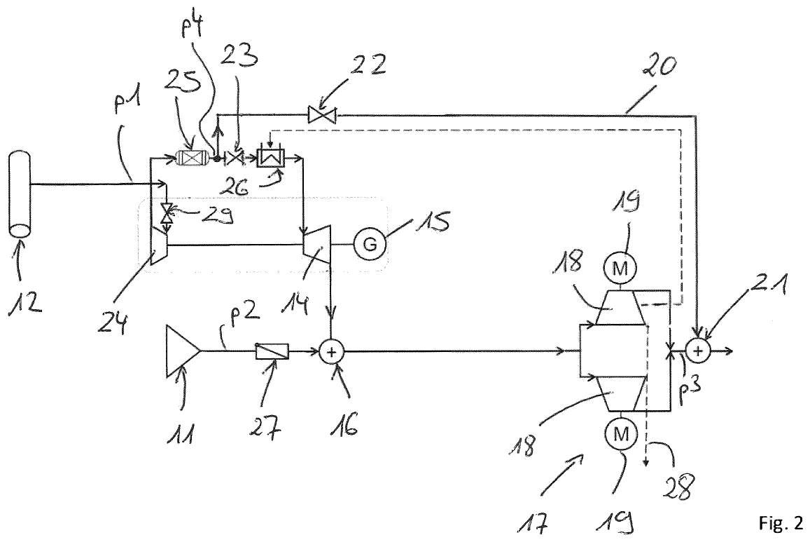

FIG. 2 shows a further development of the device 10 according to one aspect of the invention for the case in which the first pressure level p1 is significantly above the desired pressure level in the bypass line 20 and thus also significantly above the third pressure level p3. In this case, the device according to the invention then comprises a total mass flow turbine 24. The entire mass flow of at least carbon dioxide provided to the device 10 is directed upstream of the splitting device 13 by way of the total mass flow turbine 24. In FIG. 2, a scrubbing device 25 is connected between the total mass flow turbine 24 and the splitting device 13, which serves for scrubbing the carbon dioxide. A valve 29 serves for adjusting the mass flow flowing via the total mass flow turbine 24.

The splitting device 13 of FIG. 2 splits the mass flow of at least carbon dioxide provided to the device 10 likewise into a first part mass flow and a second part mass flow, however, downstream of the total mass flow turbine 24 at a pressure level p4 which is lower than the first pressure level p1.

The pressure level p4 in the region of the splitting device 13 is dependent on the desired pressure level in the bypass line 20, which is slightly above the third pressure level p3.

FIGS. 1 and 2, furthermore, show a heating device 26 for heating the first part mass flow to be conducted via the part mass flow turbine 14 directly upstream of the part mass flow turbine 14 and accordingly downstream of the splitting device 13. This heating device 26 is preferentially a heat exchanger, which is operatively connected to the compression device 17, in order to utilise in the region of the heating device 26, heat generated in the region of the compression device 17.

In the exemplary embodiments of FIGS. 1 and 2, a non-return valve 27 is connected between the hydrogen provision device 11 and the first mixing device 16. Further, FIGS. 1 and 2 show a discharge line 28 for condensate.

The exemplary embodiment of FIG. 2 differs from the exemplary embodiment of FIG. 1 also by the number of the compressors 18 in the region of the compression device 17.

The invention allows the advantageous provision of a compressed synthetic gas mixture of at least carbon dioxide and hydrogen at a pressure level, which is needed in particular by a device for producing synthetic fuel. The compressed synthetic gas mixture of at least carbon dioxide and hydrogen can be provided with low technical device expenditure and with low energy requirement.

The splitting of the total mass flow of carbon dioxide provided to the device 10 into the first part mass flow and the second part mass flow is significant, wherein the first part mass flow is conducted via the part mass flow turbine 14 and in the region of the first mixing device 16, is mixed with the hydrogen provided to the device 10. The second part mass flow is directed via the bypass line 20, past the part mass flow turbine 14 and past the compression device 17, in the direction of the second mixing device 21 and there mixed with the mixture of the first part mass flow and the hydrogen compressed in the compression device 14.

Thus, while there have shown and described and pointed out fundamental novel features of the invention as applied to a preferred aspect thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A device configured to provide a synthetic gas mixture of at least carbon dioxide and hydrogen, the device provided with a mass flow of at least carbon dioxide at a first pressure level and a mass flow of at least hydrogen at a second pressure level, comprising:
a splitting device configured to split the mass flow of at least carbon dioxide into a first part mass flow and a second part mass flow;
a part mass flow turbine configured to expand the first part mass flow of at least carbon dioxide;
a first mixing device for mixing the first part mass flow expanded by the part mass flow turbine of at least carbon dioxide with the mass flow of at least hydrogen;
a compression device configured to compress the mixture provided by the first mixing device of the at least carbon dioxide and hydrogen to a third pressure level; and
a bypass line, via which the second part mass flow of at least carbon dioxide is conducted past the part mass flow turbine, the first mixing device, and the compression device towards a second mixing device, to mix in a region of the second mixing device the second part flow of at least carbon dioxide conducted via the bypass line with the mixture compressed by the compression device.

2. The device according to claim 1, wherein
the splitting device is equipped to split the mass flow of at least carbon dioxide into the one first part mass flow and the second part mass flow in such a manner that the first part mass flow corresponds to between 25% and 75% and the second part mass flow to between 75% and 25% of an entire mass flow of at least carbon dioxide provided to the device.

3. The device according to claim 2, wherein
the first part mass flow corresponds to between 30% and 70% and the second part mass flow to between 70% and 30%, or the first part mass flow to between 40% and 60% and the second part mass flow to between 60% and 40% of the entire mass flow of at least carbon dioxide provided to the device.

4. The device according to claim 1, wherein
the splitting device comprises at least one valve.

5. The device according to claim 1, wherein
a pressure level in the bypass line is 0.1 bar to 1 bar above the third pressure level.

6. The device according to claim 5, wherein
the pressure level in the bypass line is above the third pressure level by at least one of:
0.1 bar to 0.7 bar;
0.1 bar to 0.6 bar;
0.2 bar to 0.6 bar; and
0.2 bar to 0.5 bar.

7. The device according to claim 5, wherein
a total mass flow turbine for expanding an entire mass flow of at least carbon dioxide provided to the device at the first pressure level to a pressure level dependent on the pressure level in the bypass line.

8. The device according to claim 1, further comprising:
a heating device arranged upstream of the part mass flow turbine configured to heat the first part mass flow.

9. The device according to claim 8, wherein
the heating device is a heat exchanger which utilises waste heat of the compression device to heat the first part mass flow.
